(19) United States
(12) Patent Application Publication
(10) Pub. No.: US 2026/0294272 A1
(43) Pub. Date: Oct. 1, 2026

(54) METHODS FOR DETECTING TUMOR DISEASE STATES USING CONJUGATES INCLUDING VOLATILE ORGANIC COMPOUNDS

(71) Applicant: Verily Life Sciences LLC, Dallas, TX (US)

(72) Inventors: Benjamin Krasnow, South San Francisco, CA (US); Kristopher Lavery, South San Francisco, CA (US); Sarah Sanowar, South San Francisco, CA (US); Eric Paoli, South San Francisco, CA (US)

(73) Assignee: Verily Life Sciences LLC, Dallas, TX (US)

(21) Appl. No.: 19/484,220

(22) PCT Filed: May 15, 2024
(86) PCT No.: PCT/US2024/029478

§ 371 Date: Nov. 13, 2025

Related U.S. Application Data
(60) Provisional application No. 63/466,890, filed on May 16, 2023.

(51) Int. Cl. A61B 5/08; A61B 10/00; A61K 49/00; G01N 33/497

(52) U.S. Cl. CPC A61B 5/082; A61K 49/0004; G01N 33/497; A61B 2010/0087

(57) ABSTRACT

The present disclosure is related to methods and compositions related to detecting a tumor disease state in a subject using a sample of the subject's exhaled breath. A conjugate is administered to the subject, wherein the conjugate includes a tumor targeting moiety linked to a volatile organic compound (VOC). The presence or amount of the VOC is detected as an indication of the disease state.

### CROSS-REFERENCE TO RELATED APPLICATION

This application claims the benefit of and priority to U.S. Provisional Application No. 63/466,890, filed May 16, 2023, the disclosure of which is hereby incorporated by reference in its entirety.

### TECHNICAL FIELD

The present disclosure generally relates to methods for detecting tumor disease states using volatile organic compounds (VOCs). More specifically, the present disclosure relates to compounds (e.g., conjugates) including VOCs that can be detected using breath analysis for determining the state, progression, location, or quantity of tumor cells.

### BACKGROUND OF THE INVENTION

Cancer is a leading cause of death worldwide. Many forms of cancer are only detected in very late stages leading to dramatically reduced chances of survival. Early detection of tumors or abnormal growth is a key factor in improving survival rates; however, current methods for detecting cancers require imaging and biopsy performed by a physician. These procedures can be lengthy and intrusive and require patients to be outside their homes, for example, in a hospital setting. Additionally, cancers that are found late in their development are both costly to treat and lead to poor outcomes for patients. It would be a tremendous benefit to patients to detect cancers as early as possible when their treatment is easier and has a higher chance of success. Further needed are cancer screening tests used to monitor the efficacy of treatment. To test early and often, the healthcare system needs a cancer screening test that is low cost, has a very low false positive rate, and has minimal patient burden.

Thus, a need exists for improved methods of detecting cancer in early stages and detecting therapeutic efficacy in response to treatment that are less intrusive and costly.

### SUMMARY OF THE INVENTION

The present disclosure relates to a compound that has affinity for cancerous tissue and includes a volatile organic compound (VOC). The compound can be administered via inhalation, transdermal patch, microneedles, or by other means. The compound can bind to tumor cells or react with enzymes in the tumor microenvironment or enzymes administered to the subject. The VOC can be released or retained by the compound. For example, when the compound binds to a tumor cell or reacts to an enzyme in the tumor microenvironment or administered to the subject, a linking group may be cleaved to release the VOC. The exhaled breath from the subject can be analyzed to determine the state, progression, location, or quantity of tumor cells. The conjugate including the targeting moiety linked to a VOC enables detection of many types of tumors using a breath test. Sampling breath is less invasive and more convenient than sampling blood or urine, and the test can be conducted remotely (e.g., in a non-clinical site or onsite at a pharmacy or clinic) with a breath analysis apparatus, eliminating the need to mail samples to a laboratory. This eliminates the need for tracking and handling and also eliminates sources of sample contamination and degradation.

Embodiments of the present disclosure relate to a method for detecting a tumor disease state. The method comprises contacting a tissue of interest with a conjugate including a tumor targeting moiety linked to a VOC, wherein the tumor targeting moiety has affinity and specificity for tumor cells, wherein the VOC is a non-naturally occurring VOC or a naturally occurring VOC at suitably low levels; and measuring an amount of the VOC present in an exhaled breath sample, wherein presence or amount of the VOC in exhaled breath is indicative of the presence or amount of tumor cells present at the tissue of interest. As used herein, a non-naturally occurring VOC includes a VOC that does not occur normally in healthy humans. The VOC can be but is not limited to a deuterated compound, a perfluorocarbon, or a combination thereof. The conjugate optionally comprises a linking group that links the tumor targeting moiety to the VOC. The linking group (e.g., a polyethylene glycol) can be a stable linker configured to degrade over a period of time to release the VOC. The linking group can include a cleavable linker (e.g., dimethyl disulfide).

Optionally, the method further comprises administering a secondary compound configured to cleave the linking group. The secondary compound can be configured to cleave the linker so as to separate the VOC from the tumor targeting moiety.

The method can further comprise administering a composition comprising the conjugate to a subject, wherein the composition is administered via a skin patch, a nebulizer, an inhaler, a capsule, or a microneedle system. The method can further comprise obtaining a breath sample from the subject after administration of the composition.

Measuring the amount of the volatile organic compound present in an exhaled breath sample optionally comprises providing a breath sample to a breath detector apparatus.

Also provided herein is a method for detecting a tumor disease state, wherein the method comprises administering a composition to a subject, the composition comprising a conjugate including a tumor targeting moiety linked to a VOC by a linking group; causing the volatile organic compound to separate from the conjugate; obtaining a breath sample from the subject; detecting the presence or measuring an amount of VOC separated from the conjugate; and determining the tumor disease state in the subject, wherein an amount of VOC separated from the conjugate is correlated to the tumor disease state. Causing the VOC to separate from the tumor targeting moiety optionally comprises cleaving the linking group that links the VOC to the tumor targeting moiety (e.g., chlorotoxin). The volatile organic compound can comprise deuterated ethanol or a perfluorocarbon, and the linking group can comprise polyethylene glycol (PEG) or dimethyl sulfide. The composition described herein is optionally a nasal or oral formulation, a transdermal formulation, or a microneedle formulation.

Also provided herein is a method of detecting a tumor disease state using a patient's breath, wherein the method comprises administering a composition to a subject, the composition comprising a conjugate including a tumor targeting moiety linked to a VOC; obtaining a breath sample from the patient; providing the breath sample to a breath detector apparatus including a breath VOC analyzer; measuring, using the breath VOC analyzer, an amount of the VOC. Optionally, the method further comprising determining the tumor disease state in the subject, wherein the amount of VOC separated from the compound correlate with the tumor disease state. Causing the VOC to separate from the compound optionally comprises administering a second compound to the subject that causes the VOC to be released.

Also provided is a conjugate for use in the methods disclosed herein. Thus, provided is a conjugate for detecting tumor cells, the conjugate comprising one or more VOCs (e.g., an alcohol like deuterated ethanol); a linking group (e.g., polyethylene glycol or dimethyl sulfide.) attached to a portion of the one or more VOCs; and a tumor targeting moiety (e.g., chlorotoxin) attached to a portion of the linking group; wherein the linking group is configured to be cleaved or degraded to release the VOC. Further aspects, objects, and advantages will become apparent upon consideration of the detailed description.

### BRIEF DESCRIPTION OF THE DRAWINGS

FIG. 1 shows a flowchart for a method for measuring a level of VOCs in exhaled breath.

FIG. 2 shows a flowchart for a method for determining the tumor disease state in a subject.

FIG. 3 shows a flowchart for another method for determining the tumor disease state in a subject.

### DETAILED DESCRIPTION OF SPECIFIC EMBODIMENTS

### I. Introduction

The present disclosure provides a non-invasive cancer screening method of detecting tumors at any stage of progression. In particular, the present disclosure describes a number of embodiments related to methods of detecting tumor disease states (including states of response to a therapeutic agent or therapeutic regimen) using volatile organic compounds (VOCs), conjugates including VOCs, and systems for detecting tumor disease states using VOCs. In some embodiments, the present disclosure provides a compound (e.g., a conjugate) including a tumor targeting moiety and a VOC. The compound can be provided in a composition for delivery according to multiple administration modes. For example, the composition including the compound can be administered via injection, inhalation, transdermal patch, orally, or by other means of administration. In one example, the compound including the tumor targeting moiety and the VOC can be linked by a linking group. The tumor targeting moiety is optionally configured to bind to the tumor cell or to react to molecules (e.g., enzymes) in the tumor microenvironment. Such enzymes may occur naturally in the tumor microenvironment or may be administered to the subject, optionally in the form of a tumor targeted conjugate or as an enzyme encapsulated within a nanoparticle comprising tumor targeting moieties. The conjugate can be retained at or in that tumor cell along with the VOC if the compound encounters a tumor cell. The VOC can be released (e.g., cleaved) or retained by the compound at the tumor site. For example, when the tumor targeting moiety binds to a tumor cell or reacts to a molecule present in the tumor microenvironment, the linking group may be cleaved to release the VOC. After a period of time, the subject can exhale into a breath detector apparatus, and the presence or lack of the VOC in the analyzed breath can be indicative of a state, progression, location, or quantity of tumor cells.

The present disclosure provides a non-invasive detection technique for cancer screening that provides both convenience and affordability. Although some approaches to analyzing exhaled breath have been developed to determine potential cancer biomarkers, these methods are limited to specific types cancers (e.g., lung cancer) or only detect endogenous VOCs that are identified as cancer-indicating biomarkers that are already present in the subject. However, analyzing exhaled breath for cancer-indicating biomarkers (e.g., excreted metabolic products) is difficult as thousands of endogenous VOCs have been identified in human breath. Additionally, many of these endogenous VOCs are present in exhaled breath in quantities that are less than the experimental error of the detection methods that are used to detect and/or identify them. For example, many of the VOCs in breath range from only a few parts per trillion (ppt) to a few parts per billion (ppb) concentration. Many chemical species in breath samples are at millions-fold higher concentration than prevalent VOCs, such as water vapor and carbon dioxide, which may need to be removed to avoid swamping most analytical instruments. Additionally, breath is a chemically-diverse mixture containing many forms of alcohols, ketones, and aldehydes, which complicate the identification of disease biomarkers. VOCs in breath may also include non-metabolic constituents, which may introduce false biomarkers in breath analysis.

Therefore, in order to efficiently and accurately analyze VOCs in breath to detect or identify a tumor disease state, there are many barriers to overcome. For example, if a VOC is identified that is a viable cancer-indicating biomarker, the identified VOC may need to be concentrated before analysis. General approaches to concentrating one or more VOCs of interest from dilute gaseous samples include chemical, cryogenic, and adsorptive methods. Another hurdle is identifying specific relationships between biomarker(s) and/or quantities of specific biomarkers, which can be correlated with a high level of certainty as to the presence of the disease state, with a low chance of false negatives.

The methods and systems described herein provide a novel method of determining tumor disease states by providing a conjugate including a tumor targeting moiety linked to a VOC. In some embodiments, the tumor targeting moiety can be a peptide such as chlorotoxin, which preferentially binds to tumor cells. The chlorotoxin can be linked to a VOC via a linking group. In some embodiments, the linking group can be a cleavable linker. For example, the cleavable linker can be cleaved when the tumor targeting moiety binds to a tumor cell or the cleavable linker can be cleaved when ingested by a tumor cell due to, for example, pH changes in the environment. In some embodiments, a secondary compound can be administered to cleave the linking group. For example, a peptide that alters the structure of the conjugate can release the VOC from the conjugate. In some embodiments, the linking group is not cleavable or is not cleavable for a period of time. In this embodiment, less VOCs are detected in a breath test due to retention at the tumor site. Depending on the method of separating or retaining the VOC at the tumor site, a subject can be administered a breath test. The breath test can include a VOC analyzer to measure the amount of VOCs in a patient's breath after a period of time has elapsed. The amount of VOCs in a patient's breath can be correlated to the presence of tumor, the tumor disease state, or a quantity of tumor cells.

In some embodiments, the VOC can be a non-naturally occurring VOC or an exogenous VOC. For example, the VOC can be deuterated ethanol. Deuterated ethanol is nontoxic and non-radioactive, and behaves nearly identically to ethanol. However, the extra neutrons make it distinguishable from endogenous VOCs, such as ethanol, and its presence can be traced with a high level of certainty to the administered compound because it does not naturally occur in any meaningful quantities. For example, healthy human bodies normally create low levels of ethanol and exhale it at levels in the part per billions. Therefore, the naturally occurring ethanol would cause interference with the proposed detection method if ethanol was linked to the tumor targeting moiety. Deuterated or otherwise non-naturally occurring VOCs can be linked to the tumor targeting moiety, which can be correlated with a high level of certainty to the presence of the disease state, with a low chance of false negatives.

### II. Compound Including Volatile Organic Compound

The present disclosure provides a compound including a tumor targeting moiety and a volatile organic compound (VOC). In some embodiments, the compound comprises a conjugate including a tumor targeting moiety and a volatile organic compound (VOC). In some embodiments, the tumor targeting moiety can be linked to the VOC via a linking group. For example, a linking group can be attached to a portion of the tumor targeting moiety and a portion of the VOC to link the tumor targeting moiety to the VOC. In some embodiments, the linking group can be a cleavable linker. For example, the linking group can be cleaved by a morphology change of the conjugate, a pH change (e.g., due to tumor cell absorption), or may dissolve over time. In some embodiments, the VOC is released immediately after the tumor targeting moiety binds to a tumor cell. For example, when the tumor targeting moiety binds to a tumor cell, the morphology of the conjugate may change resulting in release of the VOC. In some embodiments, the conjugate can have a general formula represented by Formula (I): X-L-N (I), wherein X is the tumor targeting moiety, L is the linking group, and N is the VOC.

In some embodiments, the tumor targeting moiety can be a peptide that preferentially binds to tumor cells. For example, the tumor targeting moiety can be chlorotoxin. Chlorotoxin is a targeting agent that directs the conjugate to a tumor cell of interest. Chlorotoxin is a highly specific marker for four grades of gliomas and tumors of neuroectodermal origin, such as medulloblastomas, neuroblastomas, ganglioneuromas, melanomas, pheochromocytomas, and small cell lung carcinomas. In some embodiments, the conjugate is a chlorotoxin conjugate. In one embodiment, the chlorotoxin conjugate includes one or more VOCs coupled to chlorotoxin via a linking group. Chlorotoxin may be native chlorotoxin, synthetic chlorotoxin, or recombinant chlorotoxin. Chlorotoxin fragments and variants having chlorotoxin binding activity (e.g., specificity and affinity to cancerous tissues) may also be used.

In some embodiments, the tumor targeting moiety comprises a molecular portion that has affinity for tumors. Known cancer treatment agents (e.g., prodrugs, small molecules, and biologics) have been studied as vehicles to target tumors. For example, fluorescently labeled chlorotoxin has been used to selectively attach fluorescent molecules to tumor sites before surgery so that all cancerous tissue is labeled for complete removal. Suitable tumor targeting moieties include tumor targeting peptides (e.g., RGD-containing peptides), proteins, antibodies (or portions thereof), lipids, aptamers, chemical compounds, or cells with pan-cancer specificity.

In some embodiments, the VOC is linked to the tumor targeting moiety (e.g., chlorotoxin) to allow for detection of the VOC of the conjugate by breath tests. Suitable VOCs are those that can be linked to the tumor targeting moiety without substantially adversely affecting the targeting and binding function of the tumor targeting moiety. Similarly, suitable VOCs retain their structure and properties after linking to a tumor targeting moiety or after the tumor targeting moiety binds to a tumor cell.

The VOC can be a synthetic or non-naturally occurring VOC. As used herein, “non-naturally occurring” VOC refers to a VOC that is not naturally produced by the subject of interest. For example, depending on the subject (e.g., humans), a VOC is selected such that low levels or no VOC of the conjugate is present. In some embodiments, a non-naturally occurring VOC can be a VOC that is not naturally formed or produced from metabolic processes in the human body. In some embodiments, the VOC can be a deuterated molecule or another non-naturally occurring VOC. For example, the VOC can be deuterated ethanol. Deuterated ethanol is non-toxic and non-radioactive, and behaves nearly identically to ethanol. However, the extra neutrons make it distinguishable from endogenous VOCs, such as ethanol, and its presence can be traced with a high level of certainty to the administered compound because it does not naturally occur in meaningful quantities. Healthy human bodies normally create low levels of ethanol and exhale it at levels in the part per billions. Therefore, the naturally occurring ethanol would cause interference with the proposed method if ethanol was linked to the tumor targeting moiety. Deuterated or otherwise non-naturally-occurring VOCs can be linked to the tumor targeting moiety, which can be correlated with a high level of certainty to the presence of the disease state, with a low chance of false-negatives. Deuterated forms of other common VOCs, such as acetone, methanol, and isopropanol may also be used. Acetone, in particular, is also a common byproduct in human breath. An additional class of safe, easily detectable VOCs are thiols, which consist of an SH endgroup connected to an aliphatic tail which may or may not contain additional functionality, such as hydroxides or ketones. Thiols are attractive due to their highly volatile nature, proven non-toxicity (they are often used as flavorings and odorants in foods and beverages), and importantly the thiol endgroup can create a cleavable disulfide bond. The cleavable disulfide linkage has been exploited in the delivery of drugs to tumor sites and can be exploited here as well to release the thiol VOC into the bloodstream and/or breath.

In some embodiments, the VOC can be a perfluorocarbon. For example, the perfluorocarbon can be perfluoropentane. Perfluoropentane is used as an injected ultrasound contrast agent and primarily exits the body via the lungs due to its inertness and high vapor pressure. Perfluoropentane is not naturally produced by humans and therefore can be correlated with a high level of certainty to the presence of the disease state, with a low chance of false negatives.

In some embodiments, the VOC can be a naturally occurring VOC that is present in the subject in less than 50 ppb, e.g., less than 10 ppb. If a VOC is used that is present in amounts greater than 50 ppb, it may cause interference with breath tests. The selected VOC, whether naturally occurring or non-naturally occurring, should not be present in high enough concentrations in food, drink, or personal hygiene products so as to cause interference with the test quantities of the VOC. Additionally, the selected VOC optionally has a low variance across test subjects (e.g., humans).

The conjugate may include a linking group that links the tumor targeting moiety to the VOC. In some embodiments, the linker can be a stable linker. Suitable stable linkers can link the tumor targeting moiety to the VOC without affecting the structure of the tumor targeting moiety or the VOC. In some embodiments, the stable linker is an oligomer. In some embodiments, the linker can be a polyethylene glycol (PEG), mono- or poly-dispersed. PEG linkers are optionally attached to the VOC by an azide, an amine, an ester, a maleimide or other attachment means. The linker may slowly degrade by natural processes to release the VOC. For example, the conjugates may concentrate over time at the location of the tumor cells. When the linker slowly degrades by natural processes, a low level of the VOC is released over a period of time. In this method, a subject, when administered the conjugate, has a fast washout period in which the subject exhales deuterated ethanol shortly after administration, then comparatively little after their body has cleared the compound. A subject with a tumor will retain more of the conjugate and have a longer washout period, exhaling deuterated ethanol at a low level for a longer time compared to a healthy person. Thus, the method is designed to administer the compound to a subject, wait a period of time, then sample the subject's breath for the presence of the VOC. The presence of the VOC would indicate the compound has been retained at the site of a tumor.

In some embodiments, the conjugate may include a cleavable linker that links the tumor targeting moiety to the VOC. The cleavable linker can be cleaved by an action or an external stimulus to separate the VOC from the tumor targeting moiety. For example, the linking group can be cleaved by a morphology change of the conjugate, a pH change (e.g., due to tumor cell absorption), or other chemical actions. In some embodiments, the cleavable linker can be dimethyl-disulfide. Dimethyl-disulfide is stable in the bloodstream but can be broken by the chemical environment inside a cell. For example, a conjugate can be brought into the cell (e.g., endocytosed) as part of chlorotoxin's affinity for cancerous cells. The VOC can be separated via chemical action on the linker, and the VOC is free to circulate, making its way to lungs and into the breath.

In some embodiments, a secondary compound can be delivered that triggers the release of the VOC from the conjugate. The secondary compound functions to separate the VOC from the conjugate. For example, the secondary compound can alter the tumor cells or trigger the VOC release through use of a co-reactant. In some embodiments, the secondary compound can be administered after the conjugate is administered. The second compound can be administered immediately or after a period of time (e.g., up to 3 days) to trigger the release of the VOC from the conjugate. For example, if the VOC is released or accelerated at low pH (<6), acidic nanoparticles attached to the conjugate could alter the tumor microenvironment, triggering immediate VOC release and improving the detectability (concentration/time) of the VOC signal.

In some embodiments, compositions that include the conjugate are provided. The composition is suitable for administration to human and animal subjects and includes a pharmaceutically acceptable carrier. The composition includes a pharmacologically effective amount of the conjugate. An effective amount can be routinely determined by established procedures. An effective amount is an amount sufficient to occupy tumor binding sites in tumor cells, but low enough to minimize non-specific binding.

In some embodiments, the conjugate of the invention may include other useful agents. Other useful agents include detectable labels and therapeutic agents. Suitable detectable labels include agents (e.g., fluorescent moieties) that provide for the detection of the conjugate by imaging methods (e.g., fluorescence imaging). Other suitable detectable labels include radiolabels (e.g., radio isotopically labeled compounds) such as ¹²⁵I, ¹⁴C, and ³¹P, among others. Suitable therapeutic agents include cytotoxic agents. Representative therapeutic agents include chemotherapeutic agents such as methotrexate, docetaxel, cisplatin, and etoposide, among others. Other therapeutic agents include nucleic acid molecules (e.g., DNAs and RNAs, such as siRNAs) for specific cancers and diseases. In some embodiments, the conjugate does not include detectable labels or therapeutic agents.

### III. Methods of Detecting a Tumor Disease State

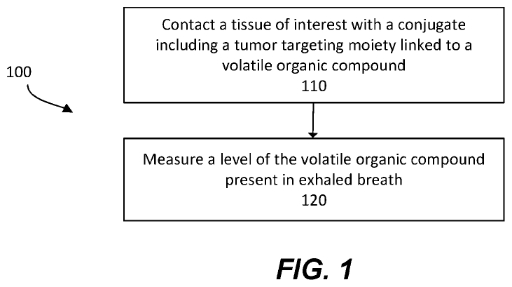

FIG. 1 provides an exemplary flow diagram of a method for detecting a tumor disease state according to some embodiments. The method 100 provides a non-invasive screening method of detecting tumors at any stage of progression or at any stage of treatment with a therapeutic agent (e.g., before a first treatment, after one or more treatments, or after completion of all treatments in a treatment regimen). The method 100 includes contacting a tissue of interest with a conjugate including a tumor targeting moiety and a volatile organic compound (VOC) 110. The tissue of interest can be any tissue in the body that is susceptible to or suspected of tumor growth. For example, the tissue can be from skin, breasts, kidney, liver, lungs, pancreas, ovaries prostate gland, head and neck. The conjugate can be administered in a composition to a subject. The composition can be administered via injection, inhalation, transdermally, orally, or by other means of administration. As the composition circulates through the body by a particular mode of administration (e.g., to the lungs via an inhaler, through the bloodstream by a skin patch, or to the intestines or colon via a capsule), the conjugate contacts and binds to tumor cells within the tissue of interest. Specifically, the tumor cells may have binding sites for the tumor targeting moiety to bind to the tumor cells. In some embodiments, the conjugate can be ingested by the tumor cell. In some embodiments, the tumor cells release unique enzymes that bind or otherwise react with the targeting moiety in the tumor milieu at or near the tissue of interest.

The method 100 includes measuring a level of the VOC present in exhaled breath 120. In some embodiments, a breath sample (e.g., exhaled breath) is provided to a breath detector apparatus including a breath analyzer. The breath analyzer can analyze the VOCs in exhaled breath that indicates the presence of the VOC in the sample. The breath analyzer is configured to determine a level of the VOC. In some embodiments in which the VOC is cleaved by molecules within the tumor microenvironment and thereby released from the conjugate, the measured amount of a VOC above a threshold amount can be indicative of the presence or an amount of tumor cells on the tissue of interest. In some embodiments in which the VOC is not cleaved from the conjugate but is instead retained at the tumor site, the measured amount of a VOC below a threshold amount can be indicative of the presence or an amount of tumor cells on the tissue of interest. For example, if the conjugate does not include a cleavable linker, the VOC would be retained at the tumor site by the conjugate and less VOC would be measured by the breath detector. In some embodiments in which the conjugate binds to the tumor and a cleaving compound is administered to the subject to cleave the linking group connecting the tumor targeting moiety to the VOC, the level of the VOC present in exhaled breath after the secondary compound cleaves the linking group indicates the presence of a tumor.

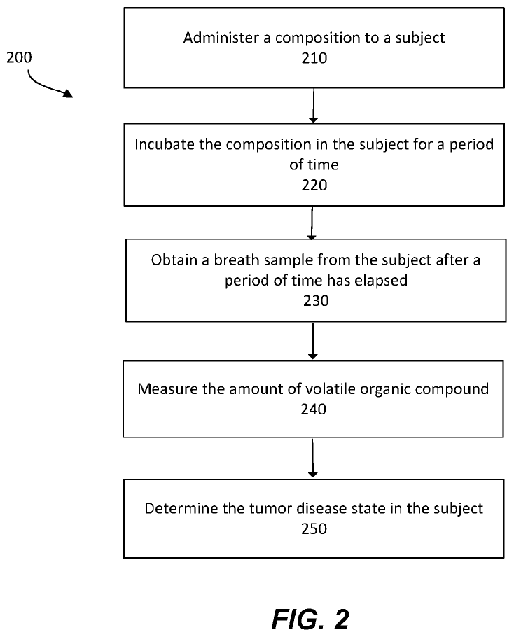

FIG. 2 provides a flow diagram of another method for detecting a tumor disease state according to some embodiments. The method 200 may include administering a composition to a subject 210. The composition can be administered via injection, inhalation, transdermal patch, orally, or by other means of administration. For example, the composition can be preloaded into a microneedle system. The microneedle can be administered to a patient to provide a predetermined dosage of the composition to a patient. In other embodiments, the composition may be administered via nasal or oral inhalation device. In some embodiments, the formulation of the composition is determined based on a location of a tumor site. For example, for lung cancer, the composition can be delivered via an oral inhalation device (e.g., an inhaler or nebulizer) for the composition to reach specific areas of the lungs.

The composition may include any of the aforementioned compounds described herein. For example, the composition may include a conjugate including a tumor targeting moiety and a VOC. In some embodiments, the tumor targeting moiety can be linked to the VOC via a linking group. For example, a linking group can be attached to a portion of the tumor targeting moiety and a portion of the VOC to link the tumor targeting moiety to the VOC. In some embodiments, the linking group can be a cleavable linker or a stable linker. For example, the linking group can be cleaved by a morphology change of the conjugate, a pH change (e.g., due to tumor cell absorption), or may dissolve over time.

The method 200 may include incubating the composition in the subject for a period of time 220. The composition including the conjugate may require sufficient time in the subject such that the tumor targeting moiety binds to a tumor cell. For example, a composition that is delivered using a transdermal patch may need sufficient time to travel to the location of the tumor site for binding to tumor cells. In some embodiments, the period of time for incubation is less than 1 hour, for example, less than 50 minutes, less than 45 minutes, less than 40 minutes, less than 30 minutes, less than 20 minutes, less than 10 minutes or less than 5 minutes. Depending on the mode of administration, the incubation time in the subject may vary. For example, an oral inhalation composition including the conjugate that is delivered to the lungs of a subject may require a short incubation time (e.g., less than 5 minutes). In contrast, a capsule composition including the conjugate may require longer incubation times for the capsule to degrade to release the conjugate at a specific location in a subject. Similarly, an injection composition including the conjugate may require longer incubation times for the conjugate to travel through the bloodstream to a tumor site.

The method 200 may include obtaining a breath sample from the subject after the period of time has elapsed 230. In some embodiments, the patient can provide a breath sample after the incubation time has elapsed. In some embodiments, the breath sample is obtained shortly after the incubation time has elapsed. In some embodiments, the breath sample is periodically obtained after the incubation time has elapsed. The method may include providing the breath sample to a breath detector apparatus that is configured to capture and hold the VOCs that is contained within the breath sample. The breath detector apparatus can analyze the VOCs in exhaled breath that indicate the presence of the VOC. The breath detector apparatus may include a breath analyzer in communication with the breath detector apparatus. The breath analyzer is configured to determine a level of the VOC.

In some embodiments, the method may include separating the VOC from the tumor targeting moiety prior to obtaining the breath sample. For example, the linking group that links the tumor targeting moiety to the VOC can be cleaved to release the VOC for detection in breath. The linking group can degrade over time to release the VOC or the linking group can be cleaved to release the VOC from the tumor targeting moiety. The linking group can be cleaved by an action or an external stimulus to separate the VOC from the tumor targeting moiety. For example, the linking group can be cleaved by a morphology change of the conjugate, a pH change (e.g., due to tumor cell absorption), or other chemical actions. In some embodiments, the linker can be dimethyl-disulfide. Dimethyl-disulfide is stable in the bloodstream but can be broken by the chemical environment inside a cell. In some embodiments, a secondary compound can be administered to cleave the linker. In some embodiments, the VOC is immediately released when a tumor targeting moiety binds to a tumor cell due to a morphology change of the conjugate.

The method 200 may include measuring an amount of VOC separated from the compound 240. For example, the breath analyzer can measure an amount of VOC separated from the compound that is present in the breath sample. The VOC analyzer may be a separate TD-GC-MS device, where the VOC of interest is captured by a sorbent tube or by a bag. The breath may be alternatively be captured by a solid phase micro-extraction (SPME) needle, a needle trap, or the like. Depending on the threshold and expected VOC concentration, a direct GC method of measurement might be employed.

The method 200 may include determining the tumor disease state in the subject 250. The amount of VOC separated from the compound is correlated to the tumor disease state. The measured level of the VOC can be compared to a predetermined threshold level of the VOC. The method may include outputting an indication of disease if the level of the VOC is more or less than the threshold level of the VOC.

In some embodiments, the method may include linking the tumor targeting moiety to the VOC using a linking group to form the conjugate. In some embodiments, the linking group can be a stable linker as described herein. In some embodiments, the stable linker can be polyethylene glycol (PEG). In some embodiments, the conjugate may include PEG as the linker, chlorotoxin as the tumor targeting moiety, and deuterated ethanol as the VOC. In this embodiment, the PEG linker may slowly degrade by natural processes to release the VOC. For example, the conjugates may concentrate over time at the location of the tumor cells. The PEG linker slowly degrades by natural processes, releasing a low level of the VOC over a long period of time. In this method, a composition including the conjugate comprising chlorotoxin linked to deuterated ethanol can be administered to a subject. For a healthy subject, the conjugate will have a fast washout period in which the subject exhales deuterated ethanol shortly after administration, then comparatively little after their body has cleared the conjugate due to the lack of binding sites for the chlorotoxin. For subject that has tumors, the tumor cells will retain more of the conjugate, and have a longer washout period, and the subject will exhale deuterated ethanol at a low level for a longer time compared to a healthy subject. Thus, the method may include administering the composition including the conjugate, incubating or waiting for a period of time, obtaining a breath sample, and detecting the presence or amount of the VOC. If the VOC is detected after a sufficient amount of time has elapsed, it would indicate that the conjugate was retained at the site of a tumor.

Also provided is a method for detecting a tumor disease state by (a) contacting a tissue of interest with a first conjugate including a tumor targeting moiety (e.g., an antibody) and a non-naturally occurring enzyme; (b) contacting the tissue with a second conjugate comprising a volatile organic compound and an enzymatic substrate, wherein the volatile organic compound is released from the second conjugate in the presence of the enzyme; and (c) measuring an amount of the released volatile organic compound present in exhaled breath, wherein the amount of the volatile organic compound in exhaled breath is indicative of an amount of tumor cells present at the tissue of interest. Antibody directed enzymatic antibody-directed enzyme prodrug therapy (ADEPT) is an example of a two stage approach of detecting a physiological marker. An antibody-enzyme conjugate targeting a target of interest is administered. After a period of time in which unbound antibody-enzyme is cleared systemically, a VOC containing conjugate is administered. This VOC containing conjugate is specifically designed to be cleaved by the enzyme attached to the antibody previously injected. Optionally the VOC-containing conjugate comprises an enzyme substrate that is specifically cleaved by the enzyme at the tumor site. Once the VOC is released, it can be detected, for example, in a sample of exhaled breath. A single enzyme can be linked to many different antibodies enabling for many different physiological targeting probes using a common enzyme-VOC conjugate pair. Alternatively, unique enzymes with unique VOCs can be used to multiplex multiple targets simultaneously.

Also provided herein is a method for detecting a tumor disease state by (a) contacting a tissue of interest with a conjugate including a non-naturally occurring volatile organic compound covalently linked to an enzymatic substrate, wherein the volatile organic compound is released from the conjugate in the presence of an enzyme; and (b) measuring an amount of the released volatile organic compound present in exhaled breath, wherein the amount of the volatile organic compound in exhaled breath is indicative of an amount of tumor cells present at the tissue of interest. The conjugate optionally further comprises a linking group between the volatile organic compound and the enzymatic substrate. The conjugate can be encapsulated by a nanoparticle comprising a tumor targeting moiety.

In some embodiments, the method may include linking the tumor targeting moiety to the VOC using a cleavable linking group to form the conjugate. In some embodiments, the cleavable linking group can be cleaved by an external stimulus, such as a cleaving agent administered to the subject. In some embodiments, the cleavable linking group can be cleaved by an internal stimulus specific to the tumor microenvironment. The linking group can be cleaved by a morphology change of the conjugate, a pH change (e.g., due to tumor cell absorption), or other chemical actions. In some embodiments, the cleavable linker can be dimethyl-disulfide. Dimethyl-disulfide is stable in the bloodstream but can be broken by the chemical environment inside a cell. In this method, a composition including the conjugate comprising chlorotoxin linked to deuterated ethanol using dimethyl disulfide can be administered to a subject. If the VOC is detected after a sufficient amount of time has elapsed (i.e., after cleavage begins to occur), it would indicate that the conjugate was retained at the site of a tumor.

In some embodiments, the method may include administering a secondary compound to the subject. The secondary compound can trigger the release of the VOC from the conjugate. For example, the secondary compound can alter the tumor cells or trigger the VOC release through use of a co-reactant. In some embodiments, the secondary compound can be administered after the conjugate is administered. The secondary compound can be administered immediately or after a period of time (e.g., up to 3 days) to trigger the release of the VOC from the conjugate. For example, if the VOC is released or accelerated at low pH (<6), acidic nanoparticles attached to the conjugate could alter the tumor microenvironment, triggering immediate VOC release.

Optionally the conjugates containing the VOC are encapsulated in a lipid nanoparticle. The encapsulated VOC-conjugates can either be targeted or designed to naturally accumulate in a target site. For example, a 100 nm nanoparticle naturally accumulates in solid tumors due to the enhanced permeability and retention effect (EPR). Also, nanoparticles containing a VOC or VOC conjugate can be actively targeted to a specific disease site using targeting ligands such as antibody fragments or peptides. Encapsulation serves to protect the VOC until it reaches its target site and provides a platform for using a single VOC or VOC-conjugate to target multiple different disease sites by changing the nanoparticle rather than the conjugate itself.

Optionally, conjugates including a VOC and a targeting moiety can be attached to the surface of a population of nanoparticles. The targeting moiety can be a peptide selected for its ability to be cleaved by a peptidases associated with a disease state. Thus, if the peptidase is present due to the disease state, the VOC will be liberated from the surface of the nanoparticle and detectable in a breath sample.

Exemplary types of cancer suitable for detection using the methods described herein are primary cancers that originate in tissues. Exemplary primary cancers include, but are not limited to supraglottic squamous cell carcinoma, pancreatic cancer, lung cancer, melanoma, colon cancer, breast cancer, renal carcinoma, prostate cancer, ovarian cancer, esophageal cancer, chondrosarcoma, cholangiocarcinoma, lymphoma, and squamous skin cancer. In other embodiments, two or more, or three or more, or four or more subject values are elevated above their respective healthy specimen values.

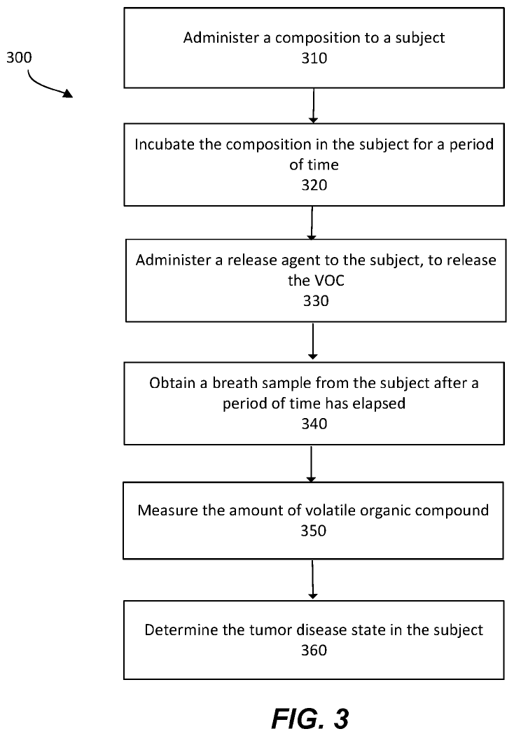

FIG. 3 provides a flow diagram of a method for detecting a tumor disease state according to some embodiments. The method 300 may include administering a composition to a subject 310, in a similar manner described for method 200.

The composition then incubates in the subject for a period of time 320, and the composition comprising a targeting moiety concentrates at the tumor site(s). Any part of the composition that does not attach to a tumor site is given enough time to wash out of the body of the subject.

A release agent may then be administered to the subject 330. The purpose of the release agent is to break the link between the targeting moiety and the VOC, either directly or by altering the local chemical environment (pH), thus releasing the VOC into the bloodstream of the subject. A breath sample is then obtained from the subject 340. This method has the benefit of first concentrating the composition on the tumor(s) and then quickly releasing the VOC, thus creating a strong signal over a relatively short duration of time (minutes to several hours), making it possible to collect a concentrated sample. The method may then measure the amount of VOC 350 to determine the tumor disease state of a subject 360.

It should be appreciated that the specific steps illustrated in FIGS. 1-3 provide a particular method of determining the tumor disease state in a subject according to some embodiments. Other sequences of steps may also be performed according to alternative embodiments. For example, alternative embodiments of the present invention may perform the steps outlined above in a different order. Moreover, the individual steps illustrated in FIGS. 1-3 may include multiple sub-steps that may be performed in various sequences as appropriate to the individual step. Furthermore, additional steps may be added or removed depending on the particular applications. One of ordinary skill in the art would recognize many variations, modifications, and alternatives.

### IV. Method of Delivery

The composition including the conjugate described herein, or the conjugate itself, can be delivered to a patient in various ways. The mode of administration can be tailored to target tumor cells in a specific region of the body. For example, the conjugate can be delivered via an oral or nasal inhaler to deliver the conjugate in the deep lungs of a subject. The conjugate can be administered in a composition to a subject. The composition can be administered via injection, inhalation, transdermally, orally, or by other means of administration. As the composition circulates through the body by a particular mode of administration (e.g., to the lungs via an inhaler or nebulizer, through the bloodstream by a syringe, or to the intestines or colon via a capsule), the conjugate contacts and binds to tumor cells on the tissue of interest. Specifically, the tumor cells may have binding sites for the tumor targeting moiety to bind to the tumor cells. In other embodiments, the conjugate can be ingested by the tumor cell.

The use of conjugates described herein take advantage of the specific enzymatic action of tumor cells to create tagged analytes which can then be detected in breath. The conjugate can use tumor-specific stimulus, such as enzymes specifically present on the surface of the tumor. The enzyme will convert the conjugate, releasing a marker-tagged side-group which can be detected as a VOC in the breath. In previous studies, these tumor cancer probes were injected into the bloodstream of the subject. This would require the presence of a clinician, which prevents rapid at-home screening.

The composition described herein can be administered via injection, inhalation, transdermally, orally, or by other means of administration. In some embodiments, the conjugate can be formulated to be delivered in an aerosol or a dry powder form. For example, the conjugate in a dry powder form can be delivered by an inhaler or nebulizer. In some embodiments, a pharmaceutical composition comprising the conjugate, or a pharmaceutically acceptable salt thereof, and an aqueous buffer, can be packaged for administration via inhalation. The compositions are suitable for inhalation that creates an aerosol suitable for deep lung inhalation. Deep lung inhalation can deliver drugs into systemic blood circulation to detect tumor states. Administration into the deep lung delivers the conjugate directly into the blood stream. This has the potential to target lung-residing tumors, for example, to detect lung cancer.

In some embodiments, the composition including the conjugate can be delivered transdermally through the skin. For example, the composition can be absorbed through the skin and into the bloodstream. In some embodiments, the composition can be provided in a dermal, mucosal, or transmucosal delivery device. The delivery device is configured to adhere to skin and may include a backer, a liner, and a reservoir. The composition can be provided in a hydrogel or an adhesive layer. In some embodiments, the composition can be sequestered in a reservoir of the device. In one embodiment, the delivery device may include a backing layer overlaying a reservoir including the composition, a cover for the reservoir having at least one opening therethrough, an adhesive layer and a liner layer. Upon removal of the liner layer, the device may be placed over the desired area of the skin or mucosa and adhesively applied thereto allowing the ingredients to flow from the reservoir through the at least one opening to the skin or mucosa. This method of delivery provides a continuous slow release of the composition for continuous monitoring. However, bolus delivery and rapid clearance or detection is also envisioned in certain embodiments.

In some embodiments, the composition can be provided in a needle system. The composition can be delivered using a microneedle system. For example, the composition can be loaded into a hollow microneedle system. In some embodiments, the composition can be a provided in a dissolvable needle. The dissolvable needle can include a polymer matrix including the conjugate for direct entry into the bloodstream. In some embodiments, the microneedle system can be administered buccally.

In some embodiments, the present disclosure provides a system for detecting a tumor disease state. The system includes an apparatus for delivering the conjugates described herein. For example, the apparatus can be a syringe including an effective amount of the composition including the conjugate described herein. The system may also include a breath detector apparatus. The breath detector apparatus may be operable to analyze VOCs that are contained within exhaled breath, and a breath analyzer in communication with the breath detector apparatus. The breath analyzer is configured to determine a level of the VOC. The apparatus may include a computer processor configured to compare the level of the VOC to a predetermined threshold level of the VOC, to output a disease signal if the level of the VOC is less than or more than the threshold level of the VOC. The apparatus alerts the user to a diagnostic condition by use of a display in communication with the computer processor, the display operable to provide an aural or visual alert of the disease indication. After collecting the VOC of interest in the breath detector apparatus, the samples may be analyzed using standard chemical analytical techniques such as Gas Chromatography Mass-Spectrometry (GC-MS), Time Of Flight Mass Spectrometry (TOF-MS) or Ion-Mobility Spectrometry (IMS). These known techniques provide knowledge on individual molecular compounds and can provide precise measures on the marker abundance in the breath samples.

The foregoing description, for purposes of explanation, used specific nomenclature to provide a thorough understanding of the described embodiments. However, it will be apparent to one skilled in the art that the specific details are not required in order to practice the described embodiments. Thus, the foregoing descriptions of specific embodiments are presented for purposes of illustration and description. They are not intended to be exhaustive or to limit the described embodiments to the precise forms disclosed. It will be apparent to one of ordinary skill in the art that many modifications and variations are possible in view of the above teachings.

1. A method for detecting a tumor disease state, comprising:
(a) contacting a tissue of interest with a conjugate including a tumor targeting moiety linked to a volatile organic compound, wherein tumor targeting moiety has affinity and specificity for tumor cells, wherein the volatile organic compound is a non-naturally occurring volatile organic compound; and
(b) measuring an amount of the volatile organic compound present in exhaled breath, wherein the amount of the volatile organic compound in exhaled breath is indicative of an amount of tumor cells present at the tissue of interest.

2. The method of claim 1, wherein the volatile organic compound comprises a deuterated compound, a perfluorocarbon, or combinations thereof.

3. The method of claim 1, wherein the conjugate comprises a linking group that links the tumor targeting moiety to the volatile organic compound.

4. The method of claim 3, wherein the linking group comprises a stable linker configured to degrade over a period of time to release the volatile organic compound.

5. The method of claim 4, wherein the stable linker comprises polyethylene glycol.

6. The method of claim 3, wherein the linking group comprises a cleavable linker, wherein the cleavable linker comprises dimethyl disulfide.

7. (canceled)

8. (canceled)

9. The method of claim 3, wherein the method further comprises administering a secondary compound configured to cleave the linking group to separate the volatile organic compound from the tumor targeting moiety.

10. The method of claim 1, further comprising administering a composition comprising the conjugate to a subject, wherein the composition is administered via a skin patch, a nebulizer, an inhaler, a capsule, or a microneedle system.

11. (canceled)

12. The method of claim 10, further comprising obtaining a breath sample from the subject after administration of the composition.

13. The method of claim 1, wherein measuring the amount of the volatile organic compound present in exhaled breath comprises providing a breath sample to a breath detector apparatus.

14. A method for detecting a tumor disease state, comprising:
(a) administering a composition to a subject, the composition comprising a conjugate including a tumor targeting moiety linked to a volatile organic compound by a linking group;
(b) causing the volatile organic compound to separate from the conjugate;
(c) obtaining a breath sample from the subject;
(d) measuring an amount of volatile organic compound separated from the conjugate; and
(e) determining the tumor disease state in the subject, wherein an amount of volatile organic compound separated from the conjugate is correlated to the tumor disease state.

15. The method of claim 14, wherein causing the volatile organic compound to separate from the tumor targeting moiety comprises cleaving the linking group that links the volatile organic compound to the tumor targeting moiety.

16. The method of claim 14, wherein the tumor targeting moiety comprises chlorotoxin.

17. The method of claim 14, wherein the volatile organic compound comprises deuterated ethanol or a perfluorocarbon.

18. The method of claim 14, wherein the linking group comprises polyethylene glycol or dimethyl sulfide.

19. The method of claim 14, wherein administering the composition comprises providing a composition as a nasal or oral formulation, a transdermal formulation, or a microneedle formulation.

20. The method of claim 14, wherein the volatile organic compound is separated from the conjugate by a releasing agent administered to the subject.

21. A method of detecting a tumor disease state using a subject's breath, comprising:
(a) administering a composition to a subject, the composition comprising a conjugate including a tumor targeting moiety linked to a volatile organic compound;
(b) obtaining a breath sample from the subject;
(c) providing the breath sample to a breath detector apparatus including a breath volatile organic compound analyzer; and
(d) measuring, using the breath volatile organic compound analyzer, an amount of the volatile organic compound.

22. The method of claim 21, further comprising determining the tumor disease state in the subject, wherein the amount of volatile organic compound measured is the amount separated from the conjugate and wherein the amount separated from the conjugate is correlated to the tumor disease state.

23. The method of claim 22, wherein the volatile organic compound is separated from the conjugate by administering a second compound that causes the volatile organic compound to be released.

24-33. (canceled)
